(19) United States
(12) Patent Application Publication
(10) Pub. No.: US 2006/0245974 A1
(43) Pub. Date: Nov. 2, 2006

(54) Method and apparatus for generating oxidized mercury having a measurable concentration

(76) Inventors: Dieter Kita, Blackstone, MA (US); James H. Grassi, Westwood, MA (US)

Correspondence Address: CHAPIN & HUANG L.L.C.;WESTBOROUGH OFFICE PARK, 1700 WEST PARK DRIVE, WESTBOROUGH, MA, 01581, US

(21) Appl. No.: 11/120,316

(22) Filed: May 2, 2005

(51) Int. Cl. B01D 53/64; G01N 33/00

(52) U.S. Cl. 422/68.1; 423/210

(57) ABSTRACT

A calibration assembly generates oxidized mercury in known concentrations for calibrating components of a mercury monitoring system. The calibrator generates an elemental mercury stream having a known elemental mercury concentration, [Hg^{0]}₁, and combines an oxidizing component with the elemental mercury stream, thereby reducing the concentration of elemental mercury [Hg⁰]₂ within the sample. The calibrator measures the concentration of elemental mercury [Hg⁰]₂ within the stream and calculates a difference between the known elemental mercury concentration, [Hg⁰]₁ and the reduced concentration [Hg⁰]₂. The difference between [Hg⁰]₁ and [Hg⁰]₂ is substantially equal to the concentration of oxidized mercury produced by the calibrator. By providing oxidized mercury at a known concentration, the calibrator allows a user to calibrate continuous emission monitoring systems for accurate response to both elemental mercury and oxidized mercury.

### FIELD OF THE INVENTION

Embodiments of the present invention relate to the generation of known concentrations of oxidized mercury and, more particularly, to the generation of known concentrations of oxidized mercury for use in calibrating a mercury monitoring system to provide accurate detection of mercury present within a fluid or gas sample.

### BACKGROUND

Emissions from fossil fuel combustion facilities, such as flue gases of coal-fired utilities and municipal solid waste incinerators, typically include mercury. The emissions include vaporized mercury as elemental mercury, Hg⁰, and/or as part of mercury- containing compounds (e.g., an oxidized form of mercury (Hg⁺²), such as in mercuric chloride or mercuric nitrate).

Many countries regulate emissions of mercury within waste gases because of potential environmental hazards posed by the mercury emissions. Hence, facilities generating gas emissions that can contain mercury typically utilize a mercury monitoring system to measure total mercury concentration in the emissions to comply with the regulations. Certain mercury monitoring systems include a converter that converts the oxidized mercury within the emissions into elemental mercury, such as by using a mercury converter performing a thermal conversion or cracking process. The mercury monitoring systems then measure the total amount or concentration of elemental mercury within the emissions using an analyzer, such as an atomic fluorescence spectrometer.

To ensure accurate measurement of the elemental mercury concentration within the emissions, the mercury monitoring systems typically include a calibration assembly. A conventional calibration assembly provides vaporized elemental mercury to the analyzer at a particular concentration. The analyzer compares the amount of elemental mercury with that of dry, substantially mercury-free gas, such as provided by a dilution gas supply. The results of the comparison allow an operator to calibrate the mercury monitoring system. SUMMARY

As described above, conventional calibration assemblies within mercury monitoring systems utilize vaporized elemental mercury to calibrate the mercury analyzer of the mercury monitoring system. However, certain mercury monitoring systems such as continuous emission monitoring systems require calibration for accurate response to both elemental mercury and oxidized mercury. And while commercial sources of elemental mercury are available for use in calibrating conventional mercury monitoring systems, reliable and accurate standards for oxidized mercury are typically not readily available for use in calibrating mercury monitoring systems.

Configurations of the present calibration assembly generate oxidized mercury in known concentrations for calibrating components (e.g., a mercury converter or mercury analyzer) of a mercury monitoring system. The calibrator generates elemental mercury having a known elemental mercury concentration, [Hg⁰]₁, and combines an oxidizing component with the elemental mercury, thereby reducing the concentration of elemental mercury to [Hg⁰]₂. The difference between [Hg⁰]₁ and [Hg⁰]₂ is substantially equal to the concentration of oxidized mercury produced by the calibrator. By providing oxidized mercury at a known concentration, the calibrator allows a user to calibrate continuous emission monitoring systems for accurate response to both elemental mercury and oxidized mercury.

In one arrangement, a mercury monitoring system calibrator includes a reactor, an elemental mercury source coupled to the reactor, an oxidizing component source coupled to the reactor, and a controller in communication with the reactor. The elemental mercury source is configured to deliver a first concentration of elemental mercury to the reactor. The oxidizing component source is configured to deliver an oxidizing component to the reactor. The reactor combines the oxidizing component with at least a portion of the elemental mercury to form an output. Based upon the difference between the first concentration of elemental mercury and the second concentration of elemental mercury within the output, the device generates a known concentration of oxidized mercury within the output. Thus the mercury monitoring system calibrator generates oxidized mercury having a known concentration. By providing oxidized mercury at a known concentration, the mercury monitoring system calibrator allows a user to calibrate continuous emission monitoring systems for accurate response to both elemental mercury and oxidized mercury.

### BRIEF DESCRIPTION OF THE DRAWINGS

The foregoing and other objects, features and advantages of the methods and apparatus will be apparent from the following description of particular embodiments, as illustrated in the accompanying drawings in which like reference characters refer to the same parts throughout the different views. The drawings are not necessarily to scale, emphasis instead being placed upon illustrating the principles of the apparatus.

FIG. 1 is a schematic of a mercury monitoring system.

FIG. 2 illustrates an arrangement of a mercury system calibrator as used within the mercury monitoring system of FIG. 1.

FIG. 3 is a flow chart of a procedure performed by the mercury system calibrator of FIG. 2 FIG. 4 is a graph illustrating detection of oxidized mercury generated by the mercury system calibrator.

FIG. 5 illustrates an arrangement of a mercury system calibrator as used within the mercury monitoring system of FIG. 1.

### DETAILED DESCRIPTION

Configurations of the present calibration assembly generate oxidized mercury in known concentrations for calibrating components (e.g., a mercury converter or mercury analyzer) of a mercury monitoring system. The calibrator generates elemental mercury having a known concentration, [Hg⁰]₁, and combines with it an oxidizing component, thereby reducing the concentration of elemental mercury to [Hg⁰]₂. The difference between [Hg⁰]₁ and [Hg⁰]₂ is substantially equal to the concentration of oxidized mercury produced by the calibrator. By providing oxidized mercury at a known concentration, the calibrator allows a user to calibrate continuous emission monitoring systems for accurate response to both elemental mercury and oxidized mercury.

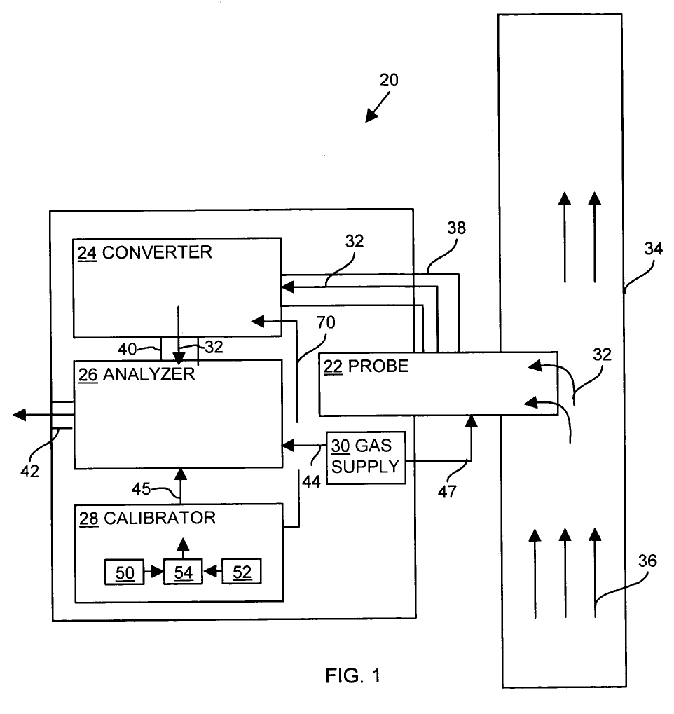

FIG. 1 illustrates a mercury monitoring system 20 for monitoring total mercury within a fluid sample, such as in an effluent gas from a coal-fired power plant, in a substantially continuous manner. The mercury monitoring system 20, or Continuous Emission Monitoring System (CEMS), includes a probe 22, a converter 24, an analyzer 26, a calibrator 28, and a dilution gas supply 30.

The probe (e.g., extraction probe) 22 is configured to receive a gas sample 32 from a sample source and deliver the gas sample 32 to the converter 24. For example, the probe 22 extends into, or is mounted proximate to, a stack or flue 34 of a coal combustion facility and collects, as the gas sample 32, a portion of the fluid or gas (e.g., effluent or emission) 36 flowing through the stack 34. The probe 22, in one arrangement, includes an inertial filter that separates particulate matter (e.g., flue ash) from the gas sample 32. Surfaces of the probe 22 that contact the gas sample 32 typically have a coating (e.g., glass) that minimizes or prevents chemical reactions between the probe 22 and mercury present within the gas sample 32.

The probe 22 is connected to the converter 24 by way of a heated conduit 38 maintained at a temperature of, for example, 150° C. The heated conduit 38 limits condensation of the gas sample 32 and “ticking” of vaporized mercury to the conduit 38 and provides efficient transport of the gas sample 32 to the converter.

The converter 24 receives the gas sample 32 from the probe 22 and is operable to convert the vapor-phase species of mercury (e.g., oxidized mercury) present within the gas sample 32 into elemental mercury and to maintain the mercury in the elemental form so as to allow the analyzer 26 to detect the total mount of mercury present within a gas sample. For example, in one arrangement, the converter 24 converts oxidized forms of mercury, Hg⁺² (e.g., HgCl₂, Hg(NO₃)₂) into elemental mercury, Hg⁰, by applying a relatively high temperature to the gas sample 32.

The analyzer 26 is connected to the converter 24 by way of a heated conduit 40 and receives the heated gas sample 32 from the converter 24. In one arrangement, the analyzer 26 is an atomic fluorescence analyzer that measures or detects an amount or a concentration of elemental mercury present within the gas sample 32. Upon completion of the detection process, the analyzer 26 exhausts the gas sample 32 to the atmosphere via an exhaust port 42.

Typically, the analyzer 26 requires periodic calibration in order to accurately detect or measure the presence of elemental mercury within a gas sample 32. Calibration is provided by the calibrator 28 which, in one arrangement is in fluid communication with the analyzer 26 through a line or conduit 45 and provides vaporized elemental mercury to the analyzer 26 at a particular concentration, such as by using a Peltier cooler/vapor pressure control and mass flow controllers. The analyzer 26 compares the amount of elemental mercury received from the calibrator 28 with that of dry, substantially mercury- free gas, received from the dilution gas supply 30 via conduit 44. The results of such a comparison allow direct calibration of the analyzer 26.

In certain cases, the analyzer 26 requires periodic calibration in order to accurately detect or measure the presence of both elemental and oxidized mercury within a gas sample 32. The calibrator 28 is connected to the converter 24 and provides a known concentration of oxidized mercury, such as in the form of a mercury-containing vapor, to the converter 24. By providing oxidized mercury having a known concentration, the calibrator 28 allows calibration of the analyzer 26 within the mercury monitoring system 20.

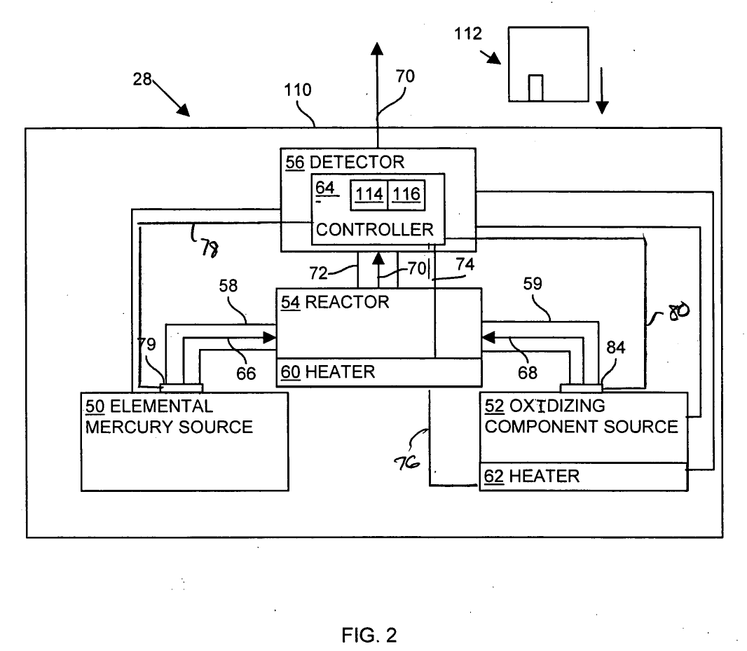

FIG. 2 illustrates an arrangement of the calibrator 28. The calibrator 28 includes an elemental mercury source 50, an oxidizing component source 52, and a reactor 54 coupled to the elemental mercury source 50 and the oxidizing component source 52.

The elemental mercury source 50 is connected to the reactor by a conduit 58 and provides a stream of elemental mercury 66, having a known concentration, to the reactor 54. For example, in one arrangement, the elemental mercury source 50 includes a vapor generator with liquid elemental mercury that evaporates from application of a particular pressure and temperature. The vapor generator further passes a flow of gas or air (e.g., substantially mercury-free gas) through the evaporated elemental mercury and delivers the vaporized mercury to the reactor 54 as a vapor stream 56 having a known (e.g., operator determined) concentration of vaporized mercury within the vapor stream. In another arrangement, the elemental mercury source 50 includes a permeation device. The permeation device contains elemental mercury in a two-phase state (liquid and gas). At a substantially constant temperature, the permeation device emits gaseous elemental mercury at a substantially constant rate through a permeable element (e.g., Teflon housing) and the elemental mercury gas 66 is delivered to the reactor 54 via the conduit 58.

The oxidizing component source 52 is connected to the reactor 54 by a conduit 59 and provides a mercury oxidizing component 68 to the reactor 54. For example, the oxidizing component source 52 provides chlorine (e.g., Cl₂) to the reactor 54 to oxidize the elemental mercury 66 received by the reactor 54. In one arrangement, the oxidizing component source 52 is configured as a container holding a chlorine generating chemical that, upon heating, generates chlorine in a gaseous phase.

In one arrangement, the oxidizing component source 52 includes a heater 62 and a mercury oxidizing component 68 such as palladium chloride (e.g., PdCl₂) or tungsten chloride in solid form. In such cases, the heater 62 increases the temperature of the palladium chloride within the oxidizing component source 52 to cause thermal separation of the palladium component from the chlorine component. The separated chlorine is then directed from the oxidizing component source 52 to the reactor 54 as chlorine gas 68.

The reactor 54 is configured to receive elemental mercury 66 from the elemental mercury source 50 and the mercury oxidizing component (e.g., chlorine) 68 from the oxidizing component source 52 and combine the oxidizing component 68 with the elemental mercury 66 to form an output or output stream 70 that includes elemental mercury gas (assuming that not all of the mercury from elemental mercury source 50 is oxidized) and mercury chloride (HgCl₂) gas. The reactor 54, in one arrangement, defines a chamber for mixing of the elemental mercury gas 66 and the chlorine gas 68 and includes a heater 60, such as a heating coil in thermal communication with the chamber. The heater 60 delivers thermal energy (e.g., heat) to the chamber to promote combining of the elemental mercury gas 66 and the chlorine gas 68 to form mercury chloride (HgCl₂).

As indicated above, the calibrator 28 generates known concentrations of oxidized mercury for calibrating continuous emission monitoring systems requiring accurate responses to both elemental mercury and oxidized mercury. The following describes an example of operation of the calibrator 28.

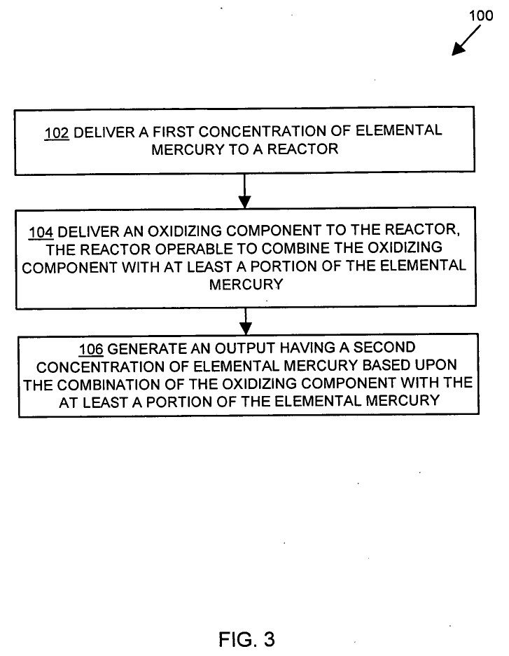

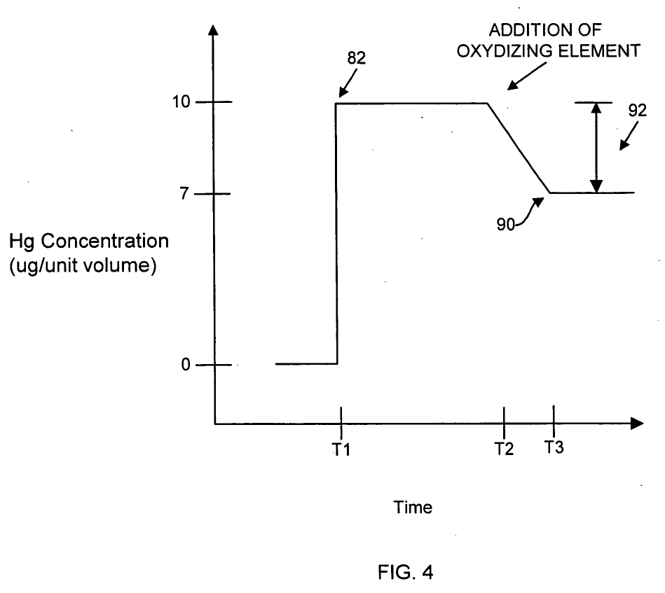

FIG. 3 is a flow chart 100 of a procedure performed by the calibrator 28 to generate a known concentration of oxidized mercury for calibration of a mercury monitoring system 20. FIG. 4, taken in conjunction with FIG. 3, illustrates a concentration of elemental mercury within the output 70 during the procedure (e.g., before and after addition of the mercury oxidizing component 68 to the elemental mercury gas 66 held by the reactor 54).

In step 102, in the calibrator 28, the elemental mercury source 50 delivers a first concentration of elemental mercury 66 to a reactor 54. For example, the elemental mercury source 50 of the calibrator 28 generates an elemental mercury stream 66 having a known or first elemental mercury concentration value, [Hg⁰]₁. As illustrated in FIG. 4, at a first time T1, the elemental mercury stream 66 (which is flowing from the elemental mercury source 50 via the conduit 58 to the reactor 54) may have a first, known concentration value 82 of 10 micrograms/unit volume.

Returning to FIG. 3, in step 104, the oxidizing component source 52 in the calibrator 28 delivers an oxidizing component 68 to the reactor 54, which may be operated at approximately room temperature (e.g., 22° C.). The reactor 54 combines the oxidizing component 68 with the elemental mercury 66. For example, as illustrated in FIG. 4, at a second time T2, the oxidizing component source 52 provides chlorine gas (e.g., C1₂) 68 to the reactor 54 as a fluid flow, carried by the conduit 59, to oxidize the elemental mercury 66 received by the reactor 54. As indicated above, the reactor 54 defines a chamber that allows for mixing of the elemental mercury (e.g., gas) 66 and the chlorine gas 68 to form mercury chloride (HgCl₂) gas. In one arrangement, the reactor receives a thermal input (e.g., heat) from the heater 60 to promote rapid combining of the chlorine gas 68 with the elemental mercury 66 to form mercury chloride (HgCl₂) gas.

Returning to FIG. 3, in step 106 the calibrator 28 generates an output 70 having a second concentration of elemental mercury (e.g., at least a portion of the elemental mercury) based upon the combination of the oxidizing component 68 with the elemental mercury 66. Since the chlorine gas 68 combines with a portion (e.g., a percentage) of the elemental mercury 66 present within the reactor to form mercury oxide gas, as illustrated in FIG. 4 in the interval between the second time T2 and a third time T3, the concentration of elemental mercury within the reactor 54 decreases from the concentration delivered to the reactor 54 from the elemental mercury source 50. For example, the concentration of elemental mercury decreases from a first concentration 82 of 10 micrograms/unit volume to a second concentration 90 of 7 micrograms/unit volume. The calibrator 28 releases the output 70 (e.g., output stream) having the second concentration 90.

Returning to FIG. 2, in one arrangement, the calibrator 28 includes a detector 56. The detector 56 is connected to the reactor 54 via a conduit 72 and is configured to receive the output stream 70 from the reactor 54. The detector 56 includes a controller 64, such as a processor 114 and a memory 116. The detector 56, such as an atomic fluorescence spectrometer, in conjunction with the controller 64, is configured to detect a concentration of elemental mercury within the output 70. For example, the detector 56 utilizes atomic fluorescence spectroscopy to measure the concentration of elemental mercury present within the reactor output 70. The detector 56 (e.g., the controller 64 of the detector 56) also compares the concentration of elemental mercury 66, [Hg⁰]₂ (e.g., the second concentration 90 of elemental mercury) present within the reactor output 70 with the known concentration of elemental mercury 66 produced by the elemental mercury source 50. The detected difference in elemental concentrations allows for the calculation of a concentration of oxidized mercury within the output 70, as described below.

For example, the detector 56 calculates a difference between the first concentration 82 of elemental mercury and the second concentration 90 of elemental mercury within the output 70 to detect a concentration of oxidized mercury within the output 70. For example, the controller 64 receives a second concentration value of the elemental mercury within the output 70 from the detector 56. The controller 64 subtracts the second, reduced elemental mercury concentration [Hg⁰]₂ from the first, known elemental mercury concentration [Hg⁰]₁. The difference between [Hg⁰]₁ and [Hg⁰]₂, illustrated in FIG. 4 as a change 92 in the elemental mercury concentration, is substantially equal to the concentration of oxidized mercury (e.g., HgCl₂) produced by the calibrator 28. By providing oxidized mercury at a measurable concentration, the calibrator 28 allows a user to calibrate continuous emission monitoring systems 20 for accurate response to both elemental mercury and oxidized mercury.

Returning to FIG. 2, in one arrangement, the controller 64 controls the thermal output of the heater 60 of the reactor 54 through an electrical line 74. The controller 64 activates the heater 60 associated with the reactor 54 to provide heat to the elemental mercury 66 and oxidizing component 68 within the reactor 54, promoting the formation of oxidized mercury. The controller 64 may also adjust the thermal output of (e.g., level of heat provided by) the heater 60 to adjust the rate of combination of the elemental mercury 66 and oxidizing component 68 and thus the concentration of oxidized mercury present within the output 70.

During operation, the controller 64 calculates the concentration of oxidized mercury within the output 70. In the case, for example, where a particular application requires the calibrator 28 to produce oxidized mercury at a particular preset concentration, the controller 64 compares a preset oxidized mercury concentration value (e.g., threshold value) with a calculated oxidized mercury value. If the preset oxidized mercury concentration value is not equal to the calculated oxidized mercury value, the controller 64 adjusts the thermal output of the heater 60 to either raise or lower the temperature of the reactor 54 (e.g., raise or lower the temperature of the elemental mercury 66 and the oxidizing component 68 within the reactor 54) so as to vary the extent of reaction of elemental mercury 66 and the oxidizing component 68, thereby adjusting the concentration of mercury oxide present within the output 70.

In one arrangement, the controller 64 is electrically connected to, and controls, the heater 62 associated with the oxidizing component source 52 through an electrical line 76. As indicated above, in one arrangement, the oxidizing component 68 contained by the oxidizing component source 52 is an oxidized metal, such as palladium chloride (i.e., PdCl₂) or tungsten chloride. During operation, the controller 64 activates the heater 62 to provide heat (e.g., the heater operates at a temperature of approximately 300° C.) to the oxidized metal, liberating chlorine gas, which flows from the oxidizing component source 52 to the reactor 54.

The controller 64, in one arrangement, is also configured to adjust a thermal output of (e.g., a level of heat provided by) the heater 62 to adjust the rate of separation of the oxidized metal into a metal component and an oxidizing component 68. By adjusting the rate of separation, the controller 64 can adjust the amount of the oxidizing component 68 delivered by the oxidizing component source 52 to the reactor 54 and thereby adjust the concentration of oxidized mercury present within the output 70.

During operation, the controller 64 calculates the concentration of oxidized mercury within the output 70. In the case, for example, where a particular application requires the calibrator 28 to produce oxidized mercury at a particular preset concentration, the controller 64 compares a preset oxidized mercury concentration value (e.g., threshold value) with a calculated oxidized mercury value. If the preset oxidized mercury concentration value is not equal to the calculated oxidized mercury value, the controller 64 adjusts the thermal output of the heater 62 to either increase or decrease the rate of separation of the oxidized metal into a metal component and an oxidizing component 68. By changing the rate of separation of the oxidized metal, the controller 64 increases or decreases the amount of the oxidizing component 68 (e.g., chlorine gas) available within the reactor 54 to chemically combine with the elemental mercury 66 within the reactor 54. As a result, the controller 64 adjusts the concentration of mercury oxide created within the reactor 54 and provided within the output 70 from the reactor 54.

In one arrangement, the controller 64 adjusts the amount of the elemental mercury 66 provided to the reactor 54 by the elemental mercury source 50 during operation. For example, in one arrangement, the controller 64 is electrically connected through an electrical line 79 to a valve 79 associated with the elemental mercury source 50 and in flow communication with the conduit 58. By increasing or decreasing the flow volume of elemental mercury 66 to the reactor 54, the controller 64 adjusts the amount of elemental mercury 66 within the reactor 54 available to chemically combine with the oxidizing component present. As a result, by adjusting the amount of the elemental mercury 66 provided to the reactor 54, the controller 64 adjusts the concentration of mercury oxide created within the reactor 54 and provided within the output 70 from the reactor 54.

For example, during operation, the controller 64 calculates the concentration of oxidized mercury within the output 70. The controller 64 compares a preset oxidized mercury concentration value (e.g., a threshold value) with the calculated oxidized mercury value. If the preset oxidized mercury concentration value is not equal to the calculated oxidized mercury value, the controller 64 adjusts (e.g., increases or decreases) the amount of the elemental mercury 66 delivered to the reactor 54, such as by adjusting the valve of the elemental mercury source 50. By adjusting the amount of the elemental mercury 66 provided to the reactor 54, the controller 64 adjusts the concentration of mercury oxide created within the reactor 54 and provided within the output 70 from the reactor 54.

In one arrangement, the controller 64 adjusts the amount of the oxidizing component 68 provided to the reactor 54 by the oxidizing component source 52 during operation. For example, in one arrangement, the controller 64 is electrically connected through an electrical line 80to a valve 84 associated with the oxidizing component source 52 and in flow communication with the conduit 59. By increasing or decreasing the flow volume of the oxidizing component 68 to the reactor 54, the controller 64 adjusts the amount of the oxidizing component 68 within the reactor 54 available to chemically combine with the elemental mercury 66 present. As a result, by adjusting the amount of the oxidizing component 68 provided to the reactor 54, the controller 64 adjusts the concentration of mercury oxide created within the reactor 54 and provided within the output 70 from the reactor 54.

For example, during operation, the controller 64 calculates the concentration of oxidized mercury within the output 70. The controller 64 compares a preset oxidized mercury concentration value (e.g., a threshold value) with the calculated oxidized mercury value. If the preset oxidized mercury concentration value is not equal to the calculated oxidized mercury value, the controller 64 adjusts (e.g., increases or decreases) the amount of the oxidizing component 68 delivered to the reactor 54, such as by adjusting the valve of the elemental mercury source 50. By adjusting the amount of the oxidizing component 68 provided to the reactor 54, the controller 64 adjusts the concentration of mercury oxide created within the reactor 54 and provided within the output 70 from the reactor 54.

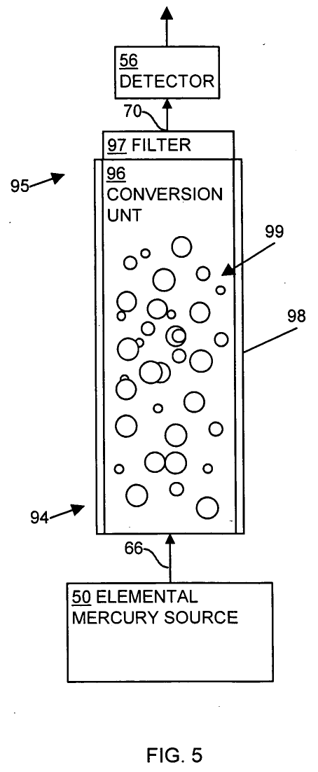

FIG. 5 illustrates an arrangement of the calibrator 28 where the reactor 54 and the oxidizing component source 52 form a single, integrated conversion unit 96. Such an arrangement minimizes the number of components required by the calibrator 28 to generate a known concentration of mercury oxide.

The conversion unit 96 has a first end 94 and a second end 95. The first end 94 is connected to the elemental mercury source 50 and is operable to direct elemental mercury 66 through the conversion unit 96 toward the second end 95. The second end 95 is connected to the detector 56 and is operable to direct an output 70 (e.g., a combination of elemental mercury and oxidized mercury in gaseous phase) toward the detector 56. The conversion unit 96 includes a filter 97 and a heater 98 and contains an oxidized metal 99, such as palladium chloride (i.e., PdCl₂).

The heater 98 is operable to heat materials within the conversion unit 96 and serves a dual purpose. First, the heater 98 is configured to increase the temperature of oxidized metal 99 within the conversion unit 96 to cause thermal separation of the metal component from the oxidizing component. Second, the heater 98 is configured to deliver thermal energy or heat to the conversion unit 96 to increase the temperature of the elemental mercury gas 66 and the oxidizing component (e.g., chlorine gas) 68 present within the conversion unit 96. Such an increase in temperature promotes combination of the elemental mercury gas 66 and the chlorine gas 68 to form mercury chloride (HgCl₂).

Returning to FIG. 2, the calibrator 28, in one arrangement, is configured as a computerized device 110. A computer program product 112 includes an application or logic instructions that are loaded into the computerized device 110 to configure the device 110 to perform as a calibrator 28.

The computerized device 110 includes the controller 64 that, in one arrangement, includes a memory 114 and a processor 116. The memory 114 can be of any type of volatile or non-volatile memory or storage system such as a computer memory (e.g., random access memory (RAM), read only memory (ROM), or another type of memory) disk memory, such as hard disk, floppy disk, optical disk, for example. The memory 114 is encoded with logic instructions and/or data that, in one embodiment of the computerized device 110, form a calibrator application configured according to embodiments of the calibrator 28. In other words, the calibrator application represents software coding instructions and/or data that reside within the memory or storage 114, or within any computer readable medium accessible to the computer device 110.

The processor 116 may be any type of circuitry or processing device such as a central processing unit, controller, application specific integrated circuit, programmable gate array, or other circuitry that can access the calibrator application encoded within the memory 114 in order to run, execute, interpret, operate, or otherwise perform the calibrator application logic instructions. In other words, in another embodiment of the computer device 110, a calibrator process represents one or more portions of the logic instructions of the calibrator application while being executed or otherwise performed on, by, or in the processor 116 within the computerized device 110.

While this invention has been particularly shown and described with reference to preferred embodiments thereof, it will be understood by those skilled in the art that various changes in form and details may be made therein without departing from the spirit and scope of the invention as defined by the appended claims.

In one example, as illustrated and described with respect to Fig.2, the detector 56 forms part of the calibrator 28. Such illustration and description is by way of example only. In an alternate arrangement, the calibrator 28 utilizes an external detector (e.g., a detector external to) the calibrator. For example, the calibrator 28 may utilize the analyzer 26 of the system 20 to perform the functions of the detector 56 described above.

FIG. 2 illustrates the detector 56 as having a single controller 64 configured to operate components of the calibrator (e.g., the elemental mercury source 50, the reactor heater 60, the oxidizing component source 52, and the oxidizing component source heater 62). Such illustration is by way of example only; in another arrangement the calibrator 28 includes separate controllers each performing one or more functions of the single controller 64 described above.

As indicated above, during operation, elemental mercury 66 flows from the elemental mercury source 50 to the reactor 54 via the conduit 58. Also during operation, the oxidizing component 68, such as chlorine gas, flows from the oxidizing component source 52 to the reactor 54 via the conduit 59. In another arrangement, the conduit 58 flows elemental mercury 66 past the oxidizing component source 52 to the reactor 54. The oxidizing component source 52 delivers the oxidizing component 68 to the reactor 54 by way of passive diffusion. Passive diffusion of the oxidizing component 68 limits or eliminates the need for a pump to force or draw the oxidizing component 68 from the oxidizing component source 52 and into the reactor 54.

FIG. 2 illustrates an arrangement of the calibrator 28 as including the elemental mercury source 50, the oxidizing component source 52, and the reactor as a single “unit”. In one arrangement, the elemental mercury source 50 and the oxidizing component source 52 are located at two separate locations. For example, the elemental mercury source 50 can be located within an instrument rack while the oxidizing component source 52 is located in or within proximity to the probe 22.

What is claimed is:

1. A method for generating a known concentration of oxidized mercury comprising:
delivering a first concentration of elemental mercury to a reactor;
delivering an oxidizing component to the reactor, the reactor operable to combine the oxidizing component with at least a portion of the elemental mercury; and
generating an output having a second concentration of elemental mercury based upon the combination of the oxidizing component with the at least a portion of the elemental mercury, the output having a known concentration of oxidized mercury based upon a difference between the first concentration of elemental mercury and the second concentration of elemental mercury.

2. The method of claim 1 comprising calculating a difference between the first concentration of elemental mercury and the second concentration of elemental mercury within the output to thereby determine a concentration of oxidized mercury within the output.

3. The method of claim 1 wherein delivering a first concentration of elemental mercury comprises delivering a first known concentration of elemental mercury to the reactor.

4. The method of claim 1 comprising heating the reactor to promote combining of the oxidizing component with the at least a portion of the elemental mercury to form the output.

5. The method of claim 4 comprising adjusting the temperature of the reactor to adjust a rate of combining of the oxidizing component and the elemental mercury.

6. The method of claim 1 wherein delivering an oxidizing component to the reactor comprises heating an oxidized metal to separate an oxidizing component from a metal component.

7. The method of claim 6 comprising adjusting a temperature of the oxidized metal to adjust a rate of separation of the oxidized metal into the metal component and the oxidizing component.

8. The method of claim 1 wherein calculating further comprises:
comparing the concentration of oxidized mercury within the output with a threshold value; and
adjusting the delivery of oxidizing component to the reactor to adjust the concentration of oxidized mercury within the output based upon the comparison.

9. The method of claim 1 wherein calculating further comprises:
comparing the concentration of oxidized mercury within the output with a threshold value; and
adjusting a volume of the elemental mercury delivered to the reactor to adjust the concentration of oxidized mercury within the output based upon the comparison.

10. A mercury monitoring system calibrator comprising:
a reactor;
an elemental mercury source in fluid communication with the reactor, the elemental mercury source configured to deliver a first concentration of elemental mercury to the reactor; and
an oxidizing component source in fluid communication with the reactor, the oxidizing component source configured to deliver an oxidizing component to the reactor, the reactor operable to combine the oxidizing component with at least a portion of the elemental mercury to form an output having a second concentration of elemental mercury within the output and having a known concentration of oxidized mercury based upon a difference between the first concentration of elemental mercury and the second concentration of elemental mercury.

11. The mercury monitoring system calibrator of claim 10 wherein the elemental mercury source is operable to deliver a known concentration of elemental mercury to the reactor as said first concentration.

12. The mercury monitoring system calibrator of claim 10 wherein the reactor comprises a heater configured to heat the reactor to promote combining of the oxidizing component with the at least a portion of the elemental mercury to form the output.

13. The mercury monitoring system calibrator of claim 11 wherein the heater is operable to adjust the temperature of the reactor to adjust a rate of combining of the oxidizing component and the elemental mercury.

14. The mercury monitoring system calibrator of claim 10 wherein the oxidizing component source comprises a heater configured to heat an oxidized metal carried by the oxidizing component source to separate an oxidizing component from a metal component.

15. The mercury monitoring system calibrator of claim 14 wherein the heater is operable to adjust the temperature of the oxidized metal to adjust a rate of separation of the oxidized metal into the metal component and the oxidizing component.

16. The mercury monitoring system calibrator of claim 10 comprising a controller in electrical communication with the reactor, the controller configured to:
detect a second concentration of elemental mercury within the output; and
calculate a difference between the first concentration of elemental mercury and the second concentration of elemental mercury within the output to detect a concentration of oxidized mercury within the output.

17. The mercury monitoring system calibrator of claim 16 wherein the controller is in electrical communication with the elemental mercury source and, when calculating, the controller is operable to:
compare the concentration of oxidized mercury within the output with a threshold value; and
adjust a volume of the oxidizing component delivered to the reactor to adjust the concentration of oxidized mercury within the output based upon the comparison.

18. The mercury monitoring system calibrator of claim 16 wherein the controller is in electrical communication with the oxidizing component source and, when calculating, the controller is operable to:
compare the concentration of oxidized mercury within the output with a threshold value; and
adjust the delivery of elemental mercury to the reactor so as to adjust the concentration of oxidized mercury within the output based upon the comparison.

19. The mercury monitoring system calibrator of claim 10 wherein the reactor and the oxidizing component source form an integral conversion unit, the elemental mercury source is in fluid communication with the conversion unit and operable to deliver a first concentration of elemental mercury to the conversion unit, and the conversion unit includes a heater for heating materials therein.

20. A computer program product having a computer-readable medium including computer program logic stored thereon that, when performed on a controller of a calibrator, causes the controller to:
deliver a first concentration of elemental mercury to a reactor;
deliver an oxidizing component to the reactor, the reactor operable to combine the oxidizing component with at least a portion of the elemental mercury to form an output;
detect a second concentration of elemental mercury within the output; and
calculate a difference between the first concentration of elemental mercury and the second concentration of elemental mercury within the output to detect a concentration of oxidized mercury within the output.
